(19) United States
(12) Patent Application Publication
(10) Pub. No.: US 2006/0110340 A1
(43) Pub. Date: May 25, 2006

(54) Compositions and methods for whitening, mineralizing and/or fluoridating calcified tissues

(75) Inventor: Ming S. Tung, Gaithersburg, MD (US)

Correspondence Address: BANNER & WITCOFF, 1001 G STREET N W, SUITE 1100, WASHINGTON, DC, 20001, US

(73) Assignee: American Dental Association Foundation, Gaithersburg, MD (US)

(21) Appl. No.: 10/996,071

(22) Filed: Nov. 24, 2004

(51) Int. Cl. A61K 8/22

(52) U.S. Cl. 424/53

(57) ABSTRACT

Kits and methods that combine whitening/stain removal of teeth with remineralization are disclosed. These kits advantageously comprise separate compositions comprising a soluble calcium salt, a soluble orthophosphate salt, and a soluble peroxide, in order to optimize the stability of these components for storage and/or the activity of these components upon use. The active ingredients may also be separated in a solid composition (e.g., a powder) or in a stable dispersion comprising a non-aqueous dispersion medium.

### FIELD OF THE INVENTION

The present invention relates to compositions and methods for whitening, mineralizing, and/or fluoridating teeth. The compositions advantageously whiten teeth while simultaneously remineralizing them to prevent and/or repair weaknesses including dental caries, exposed dentin tubules, and voids resulting from stain removal.

### BACKGROUND OF THE INVENTION

A tooth is comprised of an outer hard enamel protective layer surrounding an inner dentin layer. The outer enamel layer is naturally either opaque white or slightly off-white in color. It is composed of apatite mineral crystals that are somewhat porous. Without being bound by theory, it is believed that the porous nature of the enamel layer permits staining agents and discoloring substances to permeate into the enamel and discolor a tooth.

Dentin, the inner bony part of the tooth, contains thousands of microscopic tubules. On the crown end of the tooth, the dentin tubule ends are normally sealed by the enamel. These dentin tubules pass all the way through the dentin from the enamel-sealed crown end to the pulp chamber. On the root end of the tooth, these tubules are also sealed by a bony material called cementum. However, if either the enamel-sealed crown ends or the cementum-sealed root ends of the dentin tubules become exposed, fluid easily travels through the tubules, causing hypersensitivity and/or pain. Undesirable exposure of dentin tubules may result from mechanical abrasion, caries, chemical treatment (e.g., whitening agents), and other factors.

Plaques are a major cause of both dental decay and inflammatory periodontal disease. These plaques can contain 250 or more separate microbial species. They use sugars and other fermentable carbohydrates to produce acids, which cause demineralization of the tooth surface, and polymers, which bind the organisms themselves to the tooth surface. In its initial stages, a carious lesion is not readily apparent. However, with prolonged and repeated demineralization by plaque-created acids, a cavity will ultimately form at the lesion site.

Peroxide has been used as an oxidizing agent for whitening teeth and the treatment of various forms of stomatitis and gingivitis. See, e.g., U.S. Pat. No. 5,820,852. When applied for extended periods at high concentrations, oxidizing agents such as hydrogen peroxide and urea peroxide (carbamide) have proven effective in removing extrinsic and intrinsic stains as well as for brightening the overall shade and color of teeth. Unfortunately, the bleaching process for removing stains can simultaneously reduce the microhardness of enamel and dentin, cause post-treatment tooth sensitivity, and/or increase tooth susceptibility to demineralization. See Nathanson, D; “Vital Tooth Bleaching: Sensitivity and Pulpal Considerations,” JADA, 128 (April 1997). Hence, there is a need in the art to prevent these problems associated with tooth whitening.

Various dental products have been formulated to address plaque formation and tooth whitening. For example, U.S. Pat. No. 6,290,935 describes an oral composition comprising a peroxide first component (e.g., hydrogen peroxide, peroxydiphosphate, urea peroxide (carbamide), metal peroxides such as calcium peroxide, and salts of perborate, persilicate, perphosphate, and percarbonate) and a silicate clay activator second component, implanted with Fe ions.

U.S. Pat. No. 6,521,215 describes compositions comprising a whitening agent comprising >10% peroxide [e.g., urea peroxide (carbamide) or hydrogen peroxide] and a calcium phosphate remineralizing agent.

U.S. Pat. No. 6,221,341 describes compositions for whitening teeth and/or having antimicrobial activity. These compositions comprise an acyl group (or functionally similar group) source or precursor and a peroxide source or precursor [e.g., urea peroxide (carbamide), sodium percarbonate, sodium perborate, calcium peroxide, magnesium peroxide, or sodium peroxide], which react in an aqueous environment to generate a peroxyacid (e.g., peroxyacetic acid). The compositions have a pH>5.2 to avoid solubilizing calcium in the tooth enamel or otherwise demineralizing the tooth.

U.S. Pat. Nos. 6,102,050 and 5,967,155 describe dental flosses that administer various compounds to interproximal and subgingival dental areas for purposes such as whitening, demineralization, and desensitization. In addition to titanium dioxide as a whitening agent, other ingredients that may be incorporated into the floss include desensitizing agents, fluorides, peroxide compounds, and calcium abrading agents.

U.S. Pat. No. 5,902,568 describes compositions comprising hydrogen peroxide or a precursor and bicarbonate salt components that are maintained separate from one another to prevent their reaction prior to use.

U.S. Pat. No. 5,851,514 describes a whitening composition containing both a peroxide compound and an abrasive compound.

U.S. Pat. No. 5,698,182 describes a composition for inhibiting dental calculus and whitening teeth comprising an anticalculus phosphate salt and calcium peroxide. Also included in the composition is calcined alumina.

U.S. Pat. No. 5,648,064 describes a two component dentifrice composition comprising a peroxygen compound first component and a manganese coordination complex (e.g., manganese gluconate) second component to activate the peroxygen compound.

U.S. Pat. No. 5,624,906 describes an oral hygiene composition comprising a heteroatom containing alkyl aldonamide compound (e.g., alkoxymethyl gluconamide).

U.S. Pat. No. 6,303,104 describes an oral product for remineralizing and whitening teeth comprising a first component composed of a calcium compound and optionally a non-calcium divalent metal salt or an anhydrous calcium phosphate or dicalcium phosphate abrasive and a second component composed of an orthophosphate compound. No peroxide is mentioned.

U.S. Pat. Nos. 5,849,269; 5,820,852; and 5,776,437 describe oral compositions comprising a fluoride ion source, pyrophospate, and calcium peroxide. The oral compositions have a pH of 9.0-10.5.

There is an ongoing need in the art for compositions that whiten teeth and also remineralize them by filling voids associated with tooth decay, mechanical injury, and/or organic stain removal. There is also a need in the art for methods that achieve simultaneous whitening and remineralization of teeth in this manner. When peroxide is used as the whitening agent, its stability in an oral composition is a significant consideration. For example, most professional products containing peroxide must be stored at cold temperature and/or have limited shelf life due to peroxide degradation. However, complete stabilization of peroxide is also not a desirable objective, since the breakdown of peroxide is necessary upon use, in order to provide the free perhydroxyl anion/radical that is effective for whitening.

### SUMMARY OF THE INVENTION

The present invention is directed to kits and methods that utilize a stabilized peroxide composition to combine whitening/stain removal of teeth with remineralization. Kits of the present invention employ a soluble calcium salt, a soluble orthophosphate salt, and a soluble peroxide. These kits are based on the finding that maintaining the soluble calcium, soluble orthophosphate, and the soluble peroxide components in one or more separate environments prior to use is surprisingly beneficial for optimizing the stability and/or activity of these components. In particular, without being bound by theory, it has now been determined that (i) peroxide in solution may be stabilized in the presence of orthophosphate ion and/or in a composition at acidic pH, (ii) conversely, in order for peroxide to become activated for tooth whitening upon use, it may be destabilized by removing the soluble orthophosphate ion and/or increasing pH, and (iii) calcium orthophosphate solutions at high concentrations suitable for tooth remineralization may be stabilized at acidic pH and precipitated onto the tooth surface by increasing pH.

As an alternative means of separating a soluble calcium salt, a soluble orthophosphate salt, and a soluble peroxide prior to use, these components may be provided in a tooth whitening and remineralizing composition comprising solid particles, such as a powder composition. Otherwise, these solid particles may be dispersed in a non-aqueous dispersion medium that provides a stable dispersion. In either case, when used, these solid particles then readily dissolve in saliva and diffuse to the surface of, and within, the tooth.

Accordingly, in one embodiment, the present invention is a tooth whitening and remineralizing kit comprising a calcium-containing composition and a stabilized peroxide composition. The calcium-containing composition comprises a soluble calcium salt. The stabilized peroxide composition comprises a soluble peroxide and a soluble orthophosphate salt. In a preferred embodiment, the calcium-containing composition has an alkaline pH and the stabilized peroxide composition has an acidic pH. In another preferred embodiment, the kit further comprises a pH-control composition, where the three compositions, when combined in an aqueous environment, yield a mixture having a pH from about 5 to about 9. Preferably, the pH-control composition has an alkaline pH.

In a second embodiment, the present invention is a tooth whitening and remineralizing kit comprising a calcium-containing composition, a stabilized peroxide composition, and an orthophosphate-containing composition. The calcium-containing composition comprises a soluble calcium salt. The stabilized peroxide composition comprises a soluble peroxide and has an acidic pH. The orthophosphate-containing composition comprises a soluble orthophosphate salt, wherein the three compositions, when combined in an aqueous environment, yield a mixture having a pH from about 5 to about 9.

In a third embodiment, the present invention is a tooth whitening and remineralizing kit comprising a pH-control composition and a stabilized peroxide composition. The pH-control composition has an alkaline pH. The stabilized peroxide composition comprises a soluble peroxide and a soluble orthophosphate salt, wherein the stabilized peroxide composition has an acidic pH. Either or both of these compositions comprises a soluble calcium salt.

In a fourth embodiment, the present invention is a tooth whitening and remineralizing kit comprising a pH-control composition and a stabilized peroxide composition. The pH-control composition has an alkaline pH. The stabilized peroxide composition comprises a soluble peroxide and a calcium orthophosphate solution, wherein the stabilized peroxide composition has an acidic pH.

In any of the above embodiments, the pH-control composition preferably has a pH from about 8 to about 12 and the stabilized peroxide composition preferably has a pH from about 2 to about 6. Any of the above-mentioned compositions, and preferably the stabilized peroxide composition, may additionally contain a source of fluoride ions to promote remineralization. Also, to any of the above-mentioned compositions, excluding the stabilized peroxide composition, metal ions (e.g., Mn⁺², Fe⁺³, etc.) or other agents may be added in order to catalyze the break down of peroxide when contacted with (e.g., by mixing) the stabilized peroxide composition at or near the time of use. As stated above, the generation of the free perhydroxyl anion/radical (released upon break down of peroxide) is necessary in order to activate peroxide for tooth whitening.

In another embodiment, the present invention is a tooth whitening and remineralizing composition comprising solid particles of a soluble calcium salt, a soluble orthophosphate salt, and a soluble peroxide. In a preferred embodiment, the composition is a stable dispersion and further comprises a non-aqueous dispersion medium. In another preferred embodiment, the composition further comprises a source of fluoride ions and/or a source of carbonate ions.

In yet another embodiment, the present invention is a method for whitening and remineralizing teeth. The method comprises combining a calcium-containing composition and/or a pH-control composition, as described above, with a second composition comprising a soluble peroxide and a soluble orthophosphate salt, to yield a mixture and simultaneously or sequentially applying the mixture to the teeth. In a preferred embodiment, the method comprises applying a calcium-containing composition comprising a soluble calcium salt to the teeth and thereafter applying a second composition comprising a soluble peroxide and a soluble orthophosphate salt to the teeth. However, the calcium-containing and second compositions may also be applied in the reverse of this order. In another preferred embodiment, the method comprises combining a calcium-containing composition with a second composition that is obtained by combining a pH-control composition having an alkaline pH and a stabilized peroxide composition having an acidic pH and comprising the soluble peroxide and the soluble orthophosphate salt.

These and other embodiments of the invention are discussed in the following detailed description.

### BRIEF DESCRIPTION OF THE DRAWING

FIG. 1 shows the stability of several peroxide-containing compositions, based on measured remaining peroxide after a 54-day or 1-year storage period, as a function of the pH of these peroxide compositions.

### DETAILED DESCRIPTION OF THE INVENTION

Kits of the present invention are capable of delivering sources of calcium, orthophosphate, and peroxide to the surface of the tooth for both whitening and remineralization. Calcium ions and orthophosphate ions deposit or precipitate, as calcium orthophosphate, to remineralize the tooth, thereby strengthening it, reversing or inhibiting its decay, and/or attenuating its sensitivity. Remineralization also effectively compensates for voids left behind and increased tooth porosity resulting from the concurrent process of peroxide oxidation and removal of dental stains. The reduction in tooth porosity due to reminerization, in turn, may prevent or slow the re-formation of a new stain within the void and also prolong the whitening effect. Because the deposited calcium orthophosphate is itself white, remineralization may further enhance whitening. Also, the precipitation of calcium orthophosphate onto and/or into the tooth surface can close pores in enamel and plug dentin tubules at the crown and/or root ends to reduce or eliminate post-treatment tooth sensitivity.

Kits of the present invention comprise compositions comprising soluble components that provide sources of calcium ions, orthophosphate ions, and dissolved peroxide. Advantageously, the soluble components are provided in more than one composition, making it possible to maintain one or more of the soluble components in one environment while maintaining one or more of the other soluble components in a different environment. In particular, it has been determined that effective kits and methods for promoting tooth whitening by peroxide and tooth remineralization by calcium salts and orthophosphate salts incorporate separate compositions that exhibit enhanced stability prior to use and/or that provide enhanced reactivity on the tooth surface during use.

Importantly, it has now been determined that peroxide may be stabilized for storage, in a stabilized peroxide composition, by maintaining it in the presence of orthophosphate ion, at an acidic pH, or both. The stabilized peroxide may then become destabilized, or reactive for tooth whitening upon use, by removing the soluble orthophosphate ion (or reducing its solution concentration), and/or increasing pH. Advantageously, removal of soluble orthophosphate may be achieved by precipitating it as solid calcium orthophosphate (or tooth mineral), which beneficially results in tooth remineralization. This precipitation or remineralization can be achieved by contacting the peroxide composition, stabilized with orthophosphate ion, with calcium in solution (i.e., with calcium ions in a calcium-containing solution). The presence of the orthophosphate ion together with calcium ion will effect the desired precipitation of calcium orthophosphate, which is substantially insoluble under non-acidic conditions. As a consequence of this precipitation, soluble orthophosphate ion (used as a stabilizer during storage) is removed upon use, causing the peroxide to become destabilized or activated for tooth whitening. That is, the destabilization of peroxide upon use results in the formation of perhydroxyl anions/radicals, which are beneficial for stain removal and whitening. If desired, this destabilization may be further enhanced when the source of calcium ions (e.g., calcium present in solution in a calcium-containing composition) contains a catalytic agent (e.g., a metal ion) suitable to facilitate the break down of peroxide and consequently the formation of perhydroxyl anions/radicals. In this case, peroxide is activated by two mechanisms upon contact with calcium in solution upon use, or shortly prior to use: (1) the removal of the stabilizing agent, orthophosphate, and (2) catalytic effects.

A number of agents, if initially present in the calcium-containing composition (or other composition that is initially maintained separate from a stabilized peroxide composition), can actively catalyze the break down of peroxide. These include catalytic ions, such as metal ions, including manganese, iron, chromium, cobalt, nickel, copper, zinc, and barium. Other catalytic ions include halide anions such as chloride, bromide, and iodide are also useful for this purpose, as well as selenium. Combinations of agents may also be employed. For any of these catalytic agents, high concentrations are not required to achieve a significant increase in the rate of peroxide breakdown. In general, therefore, any of these catalysts will be effective at a concentration of 100 ppm or less, and, if used, are preferably present at a concentration from about 1 to about 100 ppm.

In a preferred embodiment, therefore, the calcium-containing composition (i.e., the source of calcium ions) of kits of the present invention comprises an agent that catalyzes the break down of peroxide. Preferably, the agent is a catalytic ion. More preferably, the agent comprises manganese and, even more preferably, comprises a combination of manganese and zinc. Zinc exhibits an unusual capacity to either stabilize, or catalyze the break down of, hydrogen peroxide.

Aside from removal of orthophosphate ion, another technique for destabilizing or activating the stabilized peroxide involves increasing its pH. This may be readily accomplished by contacting the stabilized peroxide composition with a pH-control composition, having an alkaline pH, upon use. Preferably, the stabilized peroxide composition and pH control compositions combine to yield a mixture having a pH from about 5 to about 9, which is generally tolerable in terms of taste. Preferably, the peroxide is activated using both of the above-described approaches of removing orthophosphate ion through precipitation and increasing pH. Either or both of these peroxide activation methods may be combined with the use of a catalyst, as described above.

Associated with the above findings is the provision of tooth whitening and remineralization kits comprising two or more compositions, and methods of using such kits. The first composition is a calcium-containing composition comprising a soluble calcium salt and the second composition is a stabilized peroxide composition comprising both a soluble peroxide and a soluble orthophosphate salt. Preferably, the first and second compositions have an alkaline pH and an acidic pH, respectively. In the mixture that results from combining these compositions, the soluble peroxide becomes activated by the presence of calcium ion in its environment (due to calcium phosphate precipitation, as described above), and optionally also by the increase in pH. Because combining the first and second compositions also precipitates the tooth mineral calcium orthophosphate, tooth remineralization occurs simultaneously with peroxide destabilization. In an alternate embodiment, a third pH-control composition, preferably having an alkaline pH, is used to increase the soluble peroxide pH and thereby activate it.

In another alternate embodiment, kits of the present invention comprise a calcium-containing composition comprising a soluble calcium salt and a stabilized peroxide composition comprising a soluble peroxide, which is stabilized at an acidic pH. The kit further comprises a third composition which is an orthophosphate-containing composition comprising a soluble orthophosphate salt, such that, when the three compositions are combined in an aqueous environment, they yield a mixture having a pH from about 5 to about 9. In this embodiment, the stabilized peroxide and orthophosphate-containing compositions may be mixed prior to use to yield a composition comprising a soluble peroxide and a soluble orthophosphate. In a variation of this embodiment, the third composition may also be a pH-control composition, preferably at alkaline pH and optionally comprising a soluble orthophosphate salt. If this third pH-control composition does not comprise a soluble orthophosphate salt, then this component is present in the stabilized peroxide composition.

In yet another embodiment, kits of the present invention comprise a pH-control composition having an alkaline pH. The kits further comprise a stabilized peroxide composition comprising a soluble peroxide that is stabilized at an acidic pH and in the presence of a soluble orthophosphate salt. In this embodiment, either or both of (i.e., at least one of) the pH-control composition and the stabilized peroxide composition comprises a soluble calcium salt.

It has also been determined that high concentrations of soluble calcium orthophosphate are particularly effective for tooth remineralization. In particular, calcium orthophosphate solutions, preferably having a concentration of calcium of at least about 0.06% by weight, and a concentration of orthophosphate of at least about 0.2% by weight, are preferred. Moreover, such concentrated calcium orthophosphate solutions may be maintained in a stable environment at acidic pH and precipitated onto the tooth surface for remineralization by increasing pH. Associated with this finding is the provision of other tooth whitening and remineralization kits comprising two or more compositions, and methods of using such kits. In one embodiment, for example, the first composition is a pH-control composition having an alkaline pH and the second composition is a stabilized peroxide composition comprising a soluble peroxide and a calcium orthophosphate solution, wherein the second composition has an acidic pH. The first composition may optionally comprise a soluble calcium salt to provide an additional source of calcium. The calcium orthophosphate solution (which in this embodiment is used as the source of both calcium ion and orthophosphate ion) and the soluble peroxide, are therefore both stabilized in the second composition having an acidic pH. Upon increasing the pH of this composition by combining or mixing it with the first pH-control composition having an alkaline pH, not only is the peroxide activated such that active oxygen is released for whitening, but also the calcium and orthophosphate ions are precipitated as calcium orthophosphate for remineralization.

In any of the embodiments described above, the compositions may be mixed or combined prior to application to the tooth surface, such that the combination/application steps are performed sequentially. In a preferred method of sequential application, for example, the compositions may be mixed and thereafter applied to the teeth in a tray. Alternatively, the compositions may be mixed on the teeth at the time of application (i.e., use) to the teeth in a simultaneous manner. Thus, simultaneous mixing and application includes methods wherein a first composition is applied onto the surface of the teeth (e.g., applying a solution with a cotton tip) and a second composition is then applied and combined with the applied first composition.

Simultaneous mixing and application represents a preferred method of teeth whitening and remineralization according to the present invention, wherein a first calcium-containing composition, comprising a soluble calcium salt, is first applied to the surface of the teeth, allowing the applied calcium to diffuse into the teeth. After application of the first composition, a second composition is applied to the teeth, wherein the second composition comprises a soluble peroxide and a soluble orthophosphate salt. In this manner, the peroxide and orthophosphate in the second composition become activated by diffusion and/or mixing with the calcium in the first composition, as described above. Both within the teeth as well as on the surface of the teeth, the peroxide is activated for whitening or bleaching and the calcium and orthophosphate are activated for precipitation or remineralization as calcium orthophosphate. In a preferred embodiment, the calcium-containing composition is an aqueous solution. In another preferred embodiment, the second composition is applied using a tray. In yet another preferred embodiment, the second composition has a pH from about 5 to about 9. In still another preferred embodiment, the second composition is obtained by combining a pH-control composition having an alkaline pH (preferably in gel form) with a stabilized peroxide composition (also preferably in gel form) having an acidic pH and comprising both the soluble peroxide and the soluble orthophosphate salt. Preferably, either or both of the pH-control composition and the stabilized peroxide composition comprises a soluble calcium salt.

As used herein, an alkaline pH of the pH-control composition refers to a pH value above 7, preferably from about 8 to about 12, and more preferably from about 9 to about 11. An acidic pH of the stabilized peroxide composition refers to a pH value below 7, preferably from about 2 to about 6, and more preferably from about 3 to about 5. Thus, an additional advantage associated with combining the compositions having alkaline and acidic pH is the desirability (e.g. for taste and other reasons) in general for oral products to have a pH that is near-neutral. Therefore, it is preferable that, when separate compositions are used to provide the soluble calcium salt, soluble orthophosphate salt, and soluble peroxide, these compositions will yield a near-neural pH when mixed prior to application to the tooth (i.e., in a sequential application), or when mixed during application (i.e., in a simultaneous application).

Based on the above, one approach to providing a whitening/remineralizing kit is the use of separate or distinct (e.g., layered) compositions. Alternatively, the compositions may, for example, be dispensed and mixed in a tray prior to application or mixed on the tooth surface. The compositions may be in any carrier forms (e.g., a gel form) known in the art and/or discussed hereinafter. In a preferred embodiment, at least one of the compositions (e.g., the calcium-containing composition, the pH-control composition, or the stabilized peroxide composition) is in a gel form. Gel forms are preferably aqueous gels, such as aqueous polyols, known in the art. An aqueous gel is preferred, for example, for providing a calcium orthophosphate gel solution.

As will become apparent from this disclosure, it is also possible for the kit to contain any number of compositions to provide the soluble calcium and orthophosphate salts and peroxide in a manner that will provide a stabilized source of peroxide during storage, active forms of calcium and peroxide upon use, and a near-neutral pH when mixed in an aqueous environment.

It is also possible, in accordance with the present invention, to separate the soluble calcium salt, the soluble orthophosphate salt, and peroxide prior to use by providing these components in solid particle compositions. Because these components are soluble in aqueous media, they will readily become active for whitening and remineralization, as described above, when contacted with saliva upon use or when dissolved in water or another aqueous medium before use. To facilitate their eventual dissolution in an aqueous medium and minimize any unpleasant, gritty texture, the solid particles are preferably in a powder form, having a particle size ranging preferably from about 1 to about 100 microns, and more preferably from about 10 to about 75 microns. Alternatively, a non-aqueous dispersion medium may be added to provide a stable dispersion of the solid particles, which are substantially insoluble in the dispersion medium. Sources of fluoride ions and/or carbonate ions are preferably added to such dispersions.

Suitable non-aqueous dispersion media for the solid-phase, water-soluble calcium salts, orthophosphate salts, and peroxides described herein, include non-aqueous oils, waxes, esters, alcohols, and polyols known in the art and described hereinafter in the forms of pastes, gels and varnishes. Preferred non-aqueous media that can provide stable dispersions of the solid components include oils such as vegetable oils, mineral oils, and essential oils, in addition to their higher molecular weight counterpart waxes and esters. Essential oils have antiseptic and antimicrobial effects, and include thymol, menthol, eucalyptol, and eugenol. Also preferred as non-aqueous dispersion media are varnishes, which typically comprise a natural polymer (e.g., colophony or pine resin) or synthetic polymer (e.g., a polyurethane based resin or a polymethacrylate based resin such as polymethyl methacrylate) in alcoholic solution. The solid components may also be stably dispersed in natural or synthetic gum base materials to provide a chewing gum composition. Such gum base materials include natural tree resins and latexes, as well as synthetic polymers. Examples include chicle and other polyterpenes and isoprenes, styrenes, butadienes, poly(vinyl acetate), or polyethylene

“Compositions” as used hereinafter refer to any of the individual compositions of the kit of the present invention wherein such compositions comprise one or more of the soluble calcium salt, the soluble orthophosphate salt, and the soluble peroxide. Also, it is understood that a single compound may serve as more than one source of these components. For example, an orthophosphate peroxyhydrate compound serves as both a soluble peroxide and a soluble orthophosphate salt.

“Stabilized” peroxide compositions, by utilizing orthophosphate ion and/or an acidic pH, are rendered more stable than reference compositions containing peroxide at neutral pH and in the absence of orthophosphate ion or known stabilizers. Stability is based on the rate of degradation, or conversion into other components (e.g., water and atomic or molecular oxygen) of peroxide over time.

By “Stable dispersions” refer to dispersions that are storage stable or able to maintain a substantially homogeneous composition (i.e., without separation, settling, foaming, coalescence, agglomeration, etc. of the dispersed phase), over an extended period of storage time. Preferably, stable dispersions of the present invention are storage stable for at least about 3 months, more preferably for at least about 6 months, and even more preferably for at least about 1 year.

The pH of the compositions may be maintained using any pH-adjusting compounds and buffer systems known in the art to be suitable for oral compositions. For example, compositions for oral use may be adjusted and maintained at an alkaline pH, preferably at a pH from about 8 to about 12, using hydroxide compounds (e.g., sodium hydroxide) and/or carbonate (e.g., sodium carbonate). Likewise, compositions may be adjusted and maintained at a pH from about 2 to about 6 using, for example, inorganic or organic acids including phosphoric acid, benzoic acid, and/or citric acid.

Soluble calcium and orthophosphate salts include those components that either contain calcium and orthophosphate ions or decompose when used orally to yield these ions, which in turn can remineralize the apatite crystal structure of enamel. Likewise, soluble peroxides include compounds that either contain the peroxide ion (e.g., hydrogen peroxide), or decompose to yield the peroxide ion. Soluble calcium salts refer to those having a calcium ion solubility in water such that the salt is capable of releasing at least about 800 ppm of calcium ions into water at 25° C. When exposed to saliva, soluble calcium salts therefore provide a source of calcium in sufficient concentration for the remineralization of teeth. Soluble calcium salts include, but are not limited to, calcium sulfate (e.g., plaster of paris), calcium chloride, calcium nitrate, calcium acetate, calcium bromide, calcium gluconate, calcium benzoate, calcium glycerophosphate, calcium formate, calcium fumarate, calcium lactate, calcium butyrate, calcium isobutyrate, calcium malate, calcium propionate, and calcium valerate. Preferred soluble calcium salts are calcium sulfate, calcium chloride, calcium nitrate, calcium acetate, calcium lactate, and mixtures thereof. Calcium orthophosphates include, but are not limited to, amorphous calcium phosphate (ACP), amorphous calcium phosphate fluoride (ACPF), and amorphous calcium carbonate phosphate (ACCP), which are described, for example, in U.S. Pat. No. 5,037,639.

Soluble orthophosphate salts suitable for use in the present invention have an orthophosphate ion solubility in water such that these salts are capable of releasing at least about 4000 ppm of orthophosphate ions into water at 25° C. and pH of 7.0, thus providing a source of orthophosphate for remineralization. Preferred soluble orthophosphate salts include the alkali metal, alkaline earth metal, and ammonium phosphate salts. Examples of orthophosphate salts include, but are not limited to, monosodium phosphate, disodium phosphate, trisodium phosphate, monopotassium phosphate, dipotassium phosphate, tripotassium phosphate, monoammonium phosphate, diammonium phosphate, triammonium phosphate, monocalcium phosphate monohydrate (MCMP), and monocalcium phosphate anhydrate (MCPA). Preferred orthophosphate salts are the more highly oxidized peroxyorthophosphates, having at least one peroxy substituent, which can readily dissociate to release active oxygen. Peroxyorthophosphates include peroxymonophosphates (PO₅⁻³) and diperoxymonophosphates (PO₆⁻³). Preferred peroxyorthophosphates include the alkali metal, and alkaline earth metal, and ammonium salts of peroxymonophosphate and diperoxymonophosphate. Preferred peroxymonophosphate salts include monopotassium peroxymonophosphate, dipotassium peroxymonophosphate, tripotassium peroxymonophosphate, monosodium peroxymonophosphate, disodium peroxymonophosphate, trisodium peroxymonophosphate, monoammonium peroxymonophosphate, diammonium peroxymonophosphate, and triammonium peroxymonophosphate and calcium peroxymonophosphates. Preferred diperoxymonophosphate salts include monopotassium diperoxymonophosphate, dipotassium diperoxymonophosphate, tripotassium diperoxymonophosphate, monosodium diperoxymonophosphate, disodium diperoxymonophosphate, trisodium diperoxymonophosphate, monoammonium diperoxymonophosphate, diammonium diperoxymonophosphate, and triammonium diperoxymonophosphate.

The synthesis of peroxymonophosphates may be carried out as described, for example, by Schmidlin and Massini (Ber., 43, 1910, page 1162). However, safer synthesis routes have been developed and are described, for example, in Can. J. Chem. 81:156-160 (2003) and U.S. Pat. Nos. 3,036,887 and 3,085,856. Alternative methods are described, for example, by J. E. Such, “Peroxophosphoric Acid & Peroxophosphates” in MELLOR'S COMPREHENSIVE TREATISE ON INORGANIC AND THEORETICAL CHEMISTRY, Vol. VIII (Supp III), Longmana, London (1971). These methods include (i) the hydrolysis of peroxydiphosphate in a strong acid solution, (ii) the reaction of H₄P₂O₇ with aqueous hydrogen peroxide, and (iii) the anodic oxidation of PO₄⁻³ or P₂O₇⁻⁴. Diperoxymonophosphates are less well known than peroxymonophosphates. However, they may be prepared, for example, in a similar manner, namely by the action of hydrogen peroxide on either P₂O₅ or pyrophosphoryl chloride (PO₃Cl₄).

The soluble peroxide of the present invention has a peroxide solubility in water at 25° C. such that the peroxide is capable of releasing at least about 4000 ppm of peroxide (or 2000 ppm of active oxygen, based on a 2:1 equivalency between peroxide oxygen and active oxygen generated therefrom). Soluble peroxides include, but are not limited to, hydrogen peroxide, urea peroxide (carbamide), alkali and alkaline earth metal peroxides (e.g., sodium peroxide), peroxyphosphates as described above, and peroxyhydrates as described, for example, in Z. ANORG. ALLGEM. CHEM., Vol. 177:177-226 (1928). Preferred soluble peroxides include hydrogen peroxide, urea peroxide (carbamide), salts of peroxyphosphate [including peroxymonophosphates (e.g., monosodium peroxymonophosphate and calcium peroxymonophosphates) and diperoxymonophosphates as discussed above, as well as peroxydiphosphates], as well as salts (e.g., the alkali and alkaline earth metal salts) of orthophosphate peroxyhydrate, pyrophosphate peroxyhydrate, triphosphate peroxyhydrate, and metaphosphate peroxyhydrate. Preferred peroxydiphosphate salts include monopotassium peroxydiphosphate, dipotassium peroxydiphosphate, tripotassium peroxydiphosphate, tetrapotassium peroxydiphosphate, monosodium peroxydiphosphate, disodium peroxydiphosphate, trisodium peroxydiphosphate, tetrasodium peroxydiphosphate, monoammonium peroxydiphosphate, diammonium peroxydiphosphate, triammonium peroxydiphosphate, and tetraamonium peroxydiphosphate. Preferred peroxyhydrate salts include sodium orthophosphate peroxyhydrate (e.g., Na₃PO₄.H₂O₂; Na₃PO₄.2H₂O₂; or Na₃PO₄.4H₂O₂.2H₂O) or the corresponding potassium or ammonium salts; sodium pyrophosphate peroxyhydrate (e.g., Na₄P₂O₇.2H₂O₂; Na₄P₂O₇.3H₂O₂; or Na₄P₂O₇. 2H₂O₂.8H₂O) or the corresponding potassium or ammonium salts; sodium triphosphate peroxyhydrate (e.g., Na₅P₃O₁₀.H₂O₂.5H₂O) or the corresponding potassium or ammonium salts; or sodium trimetaphosphate (sodium cyclotriphosphate) peroxyhydrate (e.g., Na₃P₃O₉.H₂O₂) or the corresponding potassium or ammonium salts. Other preferred soluble peroxide salts include peroxyborates, peroxycarbonates, and peroxysilicates, and in particular the alkali metal, some alkaline earth metal, and ammonium salts of peroxyborates, peroxycarbonates, and peroxysilicates.

In a preferred embodiment, the kits of the present invention further comprise, in addition to soluble calcium salt, soluble orthophosphate salt, and soluble peroxide, a fluoride ion source that is present in combination with any one or more of the compositions comprising the orthophosphate salt, or peroxide or is present as a separate or distinct composition. The fluoride ion source promotes or catalyzes precipitation (remineralization) of calcium orthophosphate. The fluoride ion source may be a fluoride compound including, but not limited to, sodium fluoride, potassium fluoride, zinc fluoride, stannous fluoride, zinc ammonium fluoride, sodium monofluorophosphate, sodium hexafluorosilicate, potassium monofluorophosphate, laurylamine hydrofluoride, diethylaminoethyloctoylamide hydrofluoride, 1-ethanol-2-hexadecylimidazoline dihydrofluoride, dodecyltrimethylammonium fluoride, tetraethylammonium fluoride, didecyldimethylammonium fluoride, cetylpyridinium fluoride, dilaurylmorpholinium fluoride, N-carboxymethyl-N-dodecyldiethylammonium fluoride, sarcosine stannous fluoride, glycine potassium fluoride, glycine hydrofluoride, sodium monofluorophosphate and mixtures thereof. Preferred sources of fluoride ion include sodium fluoride, potassium fluoride, sodium monofluorophosphate, and sodium hexafluorosilicate.

The compositions of the present invention, may comprise any conventional carrier. Carriers should be substantially toxicologically benign, such as polyols which include, but are not limited to, sorbitol, propylene glycol, lactitol, xylitol, polypropylene glycols, polyethylene glycols, and hydrogenated corn syrup. Whether or not a carrier is used, the compositions may contain one or more conventional additives described, for example, in U.S. Pat. Nos. 6,303,104; 6,290,935; 6,221,341; 5,902,568; and 5,624,906. Such additives include, but are not limited to, surfactants (e.g., anionic, cationic, nonionic, and zwitterionic surfactants), cosurfactants or cleansing agents, soaps, flavoring agents, sweetening agents, aroma agents, astringents, anti-plaque agents, anti-calculus agents, anti-bacterial agents (e.g., cetyl pyridinium chloride), additional preservatives and/or stabilizers, sudsing agents, humectants, thickening agents (including inorganic thickeners such as hydrated silica), binding agents or cothickeners, coloring or additional whitening agents, abrasive polishing agents, alcohols, buffering or additional pH-control agents, alkali metal halide salts, desensitizing agents, healing agents, other preventative caries agents, vitamins, amino acids, proteins, opacifiers, antibiotics, anti-enzymes, enzymes, other oxidizing agents, antioxidants, and water. When used, these additives are present in amounts that do not substantially adversely affect the desired stain removal and remineralizing capabilities as discussed above. The particular amounts of the additives used will depend upon the form of the carrier.

The carrier may be in any conventional form, including, but not limited to, a toothpaste, a prophylactic paste, a tooth polish, a tooth cleaning abrasive slurry, a gel, a professional gel, a varnish, a self-adhesive strip, a cream, a mouthwash, a pre- or post-brushing dental rinse, a dental spray, a dental cleanser, a dental floss, a dental cream, a floss wax product, a chewing gum, a lozenge, a tablet, a powder, a pumice flour, a polymeric compound, a carbonated solution, an edible food product, and the like. Preferably, the forms of the compositions are toothpastes, gels, professional gels, varnishes, self-adhesive strips, chewing gums, powders, pumice flour, dental rinses, carbonated solutions, or edible food products. These and other forms are known in the art and described in detail, for example, in U.S. Pat. Nos. 6,303,104 and 5,624,906. Varnishes have been used for sustained-release tooth fluoridation and are applicable for delivery of the whitening and remineralization compositions of the present invention as well. Tooth varnishes are generally compounds that are topically applied to teeth with a special brush, cotton, or tray and harden over a short time by contact with saliva, air, or both.

Professional products, such as professional gels, are generally intended for application under professional supervision and are known to contain higher amounts of peroxide or other whitening agents than those products designed for general consumer use. Professional gels are often applied using, for example, a tray to expose only dental surfaces and not the gums or soft tissues to the compositions. This manner of application is described, for example, in U.S. Pat. No. 6,221,341 along with various thickeners that are advantageously used in compositions for such applications. A preferred mode of application, when a gel or paste is used as a carrier form (whether or not it is considered a professional product), involves the use of such a tray or mouth guard to restrict the contact of the compositions to the teeth. In this type of application and also generally, it may be desirable to apply the various compositions in one or more repetitive steps (e.g., application of a composition comprising a readily available calcium ion source, followed by application of a composition comprising a readily available peroxide, orthophosphate ion, and fluoride ion). In this manner, the peroxide may be stabilized by the orthophosphate ion when these components are stored together at acidic pH, and the peroxide may thereafter be activated at the time of use in the presence of calcium ion, stored initially as a separate composition at alkaline pH. As described above, such an application is preferred for the present invention due to the susceptibility of the peroxide to degradation, decomposition, or reaction either when in direct contact with another component (e.g., the soluble calcium salt at alkaline pH) or when stored under conditions that are desired for the storage of another component, thereby adversely affecting the performance characteristics of the soluble peroxide.

The carrier may be in the form of a paste or gel, where the carrier is used in a multi-layer delivery system with two or more distinct compositions contained in layers. In this manner, it is also possible to obtain a striped product, wherein colorants of contrasting colors are incorporated in the distinct layer compositions, with the colorants being pharmacologically and physiologically non-toxic in the amounts used. Colorants used for this particular embodiment include both pigments and dyes, as described, for example, in U.S. Pat. No. 6,290,935. The use of colorants for distinct composition layers is not only aesthetically pleasing in many cases but also, where it is desired to mix the compositions thoroughly before use, provides a visual indication of how uniformly mixed the compositions are.

With respect to the various carrier forms described above, a plurality of conventional packaging methods and dispensing containers may be employed and are described, for example, in U.S. Pat. No. 6,303,104. Preferred dispensing containers include tubes, pumps, syringes, and other containers suitable for dispensing a paste, gel, liquid or dispersion (e.g., a slurry). Another preferred dispensing system is provided in U.S. Pat. No. 5,902,568, which discloses a pump dispenser having compartments for separately storing components and a means for combining the components as unmixed streams prior to use. In another embodiment, the dispensing container may separate the carrier from the active ingredients and may additionally combine these shortly before use.

Compositions of the present invention may optionally contain one or more chelating agent stabilizers (in addition to the orthophosphate or calcium orthophosphate solution), for example those described in U.S. Pat. No. 6,221,341. Such stabilizers include, but are not limited to, pharmaceutically acceptable chelating agents such as the various amino carboxylate compounds that have the capacity to form metal-ligand complexes with one or more transition metal ions in solution. These amino carboxylates include ethylenediaminetetraacetic acid (EDTA) and diethyltriaminepentaacetic (DTPA), 1,2-bis(2-aminophenoxy)ethane-N,N,N′,N′-tetraacetic acid (BAPTA), ethylene glycol-bis(2-aminoethyl)-N,N,N′,N′-tetraacetic acid (EGTA), and other amino carboxylate compounds having one or multiple carboxylate groups. Any derivative salt form of these amino carboxylate chelating agents, for example the disodium salt form, may be also used, provided that some capacity remains for the chelating agent to complex with various undesired species. Forms of these chelating agents other than salt forms are also effective and include the various ester, anhydride, and halogenated forms of these compounds. A preferred stabilizer is EDTA.

Preferably, the soluble calcium salt in compositions of the present invention is present in an amount, based on calcium, from about 0.08% to about 10% by weight; the soluble orthophosphate salt is present in an amount, based on orthophosphate equivalents, from about 0.4% to about 25% by weight; and the soluble peroxide is present, based on perhydroxyl anion (O₂⁻²) equivalents, from about 0.4% to about 35% by weight. If used, the fluoride source is present in an amount, based on fluorine, from about 10 ppm to about 0.5% by weight. If used in the calcium-containing composition of kits of the present invention, manganese or zinc is preferably present in an amount from about 1 ppm to about 100 ppm in solution. If manganese and zinc are both used in this composition, their combined amount is preferably within this range. The carrier and additives, if used, are generally present in a combined amount from about 10% to about 95% by weight.

All references cited in this specification, including without limitation, all papers, publications, patents, patent applications, presentations, texts, reports, manuscripts, brochures, books, journal articles, periodicals, and the like, are hereby incorporated by reference into this specification in their entireties. The discussion of the references herein is intended merely to summarize the assertions made by their authors and no admission is made that any reference constitutes prior art. Applicant reserves the right to challenge the accuracy and pertinence of the cited references. In view of the above, it will be seen that several advantages of the invention are achieved and other advantageous results obtained.

As various changes could be made in the above methods and compositions without departing from the scope of the invention, it is intended that all matter contained in this application, including all theoretical mechanisms and/or modes of interaction described above, shall be interpreted as illustrative only and not limiting in any way the scope of the appended claims.

The following examples are set forth as representative of the present invention. These examples are not to be construed as limiting the scope of the invention as these and other equivalent embodiments will be apparent in view of the present disclosure and appended claims.

### EXAMPLE 1

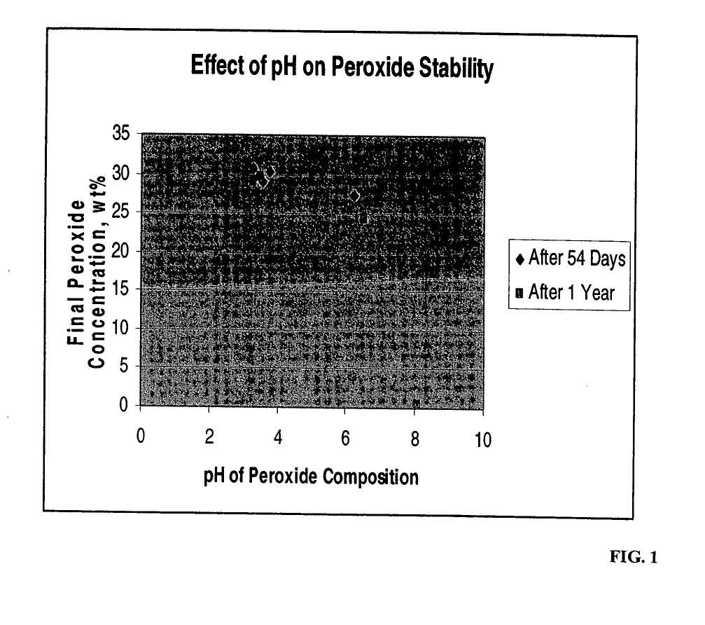

To study the effect of pH in the presence of calcium and/or phosphate on the stability of peroxide, compositions containing calcium or orthophosphate were prepared having an initial peroxide concentration of 31% by weight. The four compositions were made at various pH levels. The compositions were sampled for peroxide content to determine the extent of peroxide degradation after either 54 days or one year, or at both time periods. Results are presented below in Table 1 and also graphically in FIG. 1.

TABLE 1
pH and % Peroxide by Weight of Peroxide-Containing Compositions

| | 54 days | | one year | |
|---|---|---|---|---|
| | pH | peroxide % | pH | peroxide % |
| Control | 3.76 | 30.3 | 3.48 | 29.7 |
| Calcium-Containing | 3.55 | 29 | | |
| Phosphate-Containing | 3.25 | 30.8 | 3.28 | 29 |
| Phosphate-Containing | 6.25 | 27.3 | 6.51 | 24.4 |
| Phosphate-Containing | | | 8.1 | 0.5 |

These results indicate that pH values of about 6 and less are preferred for long-term storage of the peroxide component.

The stability of urea peroxide (i) alone, (ii) in the presence of a calcium-containing component, and (iii) in the presence of a monobasic phosphate, was studied. Compositions having an initial urea peroxide concentration of 7.5% by weight were prepared in each case. To evaluate the rate of peroxide degradation, the compositions were sampled for peroxide after 5 days and after one year. Results are presented below in Table 2.

TABLE 2
pH and % Peroxide by Weight of Urea Peroxide-Containing Compositions

| | |
|---|---|
| 5 days | One year |

| | peroxide % | pH | peroxide % |
|---|---|---|---|
| Control | 7.4 | 8.79 | 1.3 |
| Calcium-Containing | 7.5 | 8.56 | 0.6 |
| Phosphate-Containing | 7.2 | 7.89 | 6.0 |

These results indicate that monobasic phosphate is an effective stabilizer of urea peroxide for long-term storage.

### EXAMPLE 2

A tooth whitening/remineralization kit comprising separate compositions was prepared. The first peroxide and orthophosphate ion-containing gel composition had a pH of 2.8, adjusted using phosphoric acid, and the second calcium ion-containing gel composition had a pH of 11, adjusted using sodium hydroxide. The compositions were designed to provide tooth whitening from the peroxide component and tooth remineralization by the calcium and orthophosphate components, upon mixing of the compositions either prior to or during application to tooth surfaces. The components of the separate compositions and their approximate concentrations were as shown in Table 2.

TABLE 2
Peroxide and Orthophosphate Ion-Containing Gel and
Calcium Ion-Containing Gel Compositions

| | |
|---|---|
| Ingredient | Wt. % |

Peroxide and Orthophosphate Ion-Containing Gel Composition

| | |
|---|---|
| Water | 30 |
| Sodium Dihydrogen Phosphate | 15 |
| Polyol (propylene glycol, glycerol, sorbitol) | 15 |
| Hydrogen Peroxide | 15 |
| Polysorbate 65 | 15 |
| Carboxymethylcellulose | 7 |
| Sodium Lauryl Sulfate | 3 |

Adjusted to pH 2.8 with phosphoric acid

Calcium Ion-Containing Gel Composition

| | |
|---|---|
| Water | 35 |
| Polyol (propylene glycol, glycerol, sorbitol) | 20 |
| Calcium Chloride | 15 |
| Polysorbate 65 | 10 |
| Abrasives (silica, titanium dioxide) | 9 |
| Carboxymethylcellulose | 7 |
| Sodium Lauryl Sulfate | 3 |
| Flavor | 1 |

Adjusted to pH 11 with sodium hydroxide

### EXAMPLE 3

In an in vitro laboratory experiment, a solution containing 1.5 mol/L of calcium chloride was applied to a tooth surface with a cotton tip applicator or a cotton swab, followed by the application of a gel containing 1 mol/L of orthophosphate, 1000 ppm of fluoride and 21% of urea peroxide (carbamide) with a cotton tip applicator or in a tray. Measurements of whitening effectiveness indicated that this two-step application was comparable in whitening to the control gel containing 21% urea peroxide (carbamide) only. Measurements of dentin permeability indicated that this two-step application significantly decreased the dentin permeability (as measured by the rate of fluid filtration through dentin discs). These results demonstrated the effectiveness of (i) calcium orthophosphate in obstructing the dentin tubules in the presence of peroxide, as well as (ii) urea peroxide (carbamide) in whitening teeth in the presence of calcium orthophosphate, when used in the procedures described above.

### EXAMPLE 4

The calcium chloride solution described in Example 3 is applied to the tooth surface, followed by application of a mixture of the calcium ion-containing gel composition and the peroxide and orthophosphate ion-containing gel composition of Example 2.

### EXAMPLE 5

Chewing gum is prepared containing calcium chloride and sodium peroxymonophosphate as active ingredients. The gum is prepared with and without fluoride. The active ingredients are separated prior to use by being dispersed as solid particles in a conventional non-aqueous gum base.

### EXAMPLE 6

Substantially non-aqueos powders and pastes comprising soluble calcium salts, soluble peroxides, and soluble orthophosphate salts, with or without fluoride or carbonate salts, are prepared as solid compositions or dispersions. When applied to the teeth, these solid compositions or dispersions release calcium, orthophosphate, fluoride, and peroxide for remineralization and whitening of teeth. A representative powder or paste comprises 0.3 grams of calcium chloride dihydrate, 0.7 grams of dipotassium hydrogen phosphate trihydrate, 0.3 grams of urea peroxide (carbamide), and 0.0013 grams of sodium fluoride.

### EXAMPLE 7

Varnish is a good vehicle for sustained release of calcium, orthophosphate, fluoride, and peroxide. A varnish is formulated for remineralization and whitening, having, as active ingredients, calcium chloride (2%), sodium peroxymonophosphate (4%), and sodium fluoride (4%). These active ingredients are dispersed in a non-aqueous medium that is a varnish comprising resin (56%), alcohol (21%), ceytlpyridinium chloride (3%), polymethacrylates (8.5%), and polyethylene glycol (1.5%). The varnish may be painted onto the tooth with a brush, cotton pellet, or other applicator and adheres to the tooth to provide a continuing release of the active ingredients for several hours, thereby promoting remineralization and whitening.

What is claimed is:

1. A tooth whitening and remineralization kit comprising:
(a) a calcium-containing composition comprising a soluble calcium salt, and
(b) a stabilized peroxide composition comprising a soluble peroxide and a soluble orthophosphate salt.

2. The kit of claim 1, wherein said calcium-containing composition further comprises an agent that catalyzes the break down of peroxide, wherein said agent is a catalytic ion.

3. The kit of claim 1, wherein said calcium-containing composition has an alkaline pH.

4. The kit of claim 3, wherein said calcium-containing composition has a pH from about 8 to about 12.

5. The kit of claim 1, wherein said stabilized peroxide composition has an acidic pH.

6. The kit of claim 5, wherein said stabilized peroxide composition has a pH from about 2 to about 6.

7. The kit of claim 5, further comprising:
(c) a pH-control composition having an alkaline pH,
wherein said calcium-containing composition, said stabilized peroxide composition, and said pH-control composition, when combined in an aqueous environment, yield a mixture having a pH from about 5 to about 9.

8. A tooth whitening and remineralizing kit comprising:
(a) a calcium-containing composition comprising a soluble calcium salt,
(b) a stabilized peroxide composition comprising a soluble peroxide and having an acidic pH, and
(c) a orthophosphate-containing composition comprising a soluble orthophosphate salt,
wherein said calcium-containing composition, said stabilized peroxide composition, and said orthophosphate-containing composition, when combined in an aqueous environment, yield a mixture having a pH from about 5 to about 9.

9. A tooth whitening and remineralizing kit comprising:
(a) a pH-control composition having an alkaline pH;
(b) a stabilized peroxide composition comprising a soluble peroxide and a soluble orthophosphate salt, wherein said stabilized peroxide composition has an acidic pH,
wherein at least one of said pH-control composition and said stabilized phosphate composition comprises a soluble calcium salt.

10. A tooth whitening and remineralizing kit comprising:
(a) a pH-control composition having an alkaline pH, and
(b) a stabilized peroxide composition comprising a soluble peroxide and a calcium orthophosphate solution, wherein said stabilized peroxide composition has an acidic pH.

11. The kit of claim 10, wherein said pH-control composition is in a gel form that is pH-adjusted with a hydroxide compound or a carbonate compound.

12. The kit of claim 10, wherein said stabilized peroxide composition is in a gel form having said calcium orthophosphate dissolved therein.

13. The kit of claim 1, wherein said soluble calcium salt is selected from the group consisting of:
(a) calcium sulfate;
(b) calcium chloride;
(c) calcium nitrate;
(d) calcium acetate;
(e) calcium lactate; and
(f) combinations thereof.

14. The kit of claim 1, wherein said soluble orthophosphate salt is selected from the group consisting of:
(a) a peroxyorthophosphate;
(b) monosodium phosphate;
(c) disodium phosphate;
(d) trisodium phosphate;
(e) monopotassium phosphate;
(f) dipotassium phosphate;
(g) tripotassium phosphate;
(h) monoammonium phosphate;
(i) diammonium phosphate;
(j) triammonium phosphate;
(k) monocalcium phosphate monohydrate (MCMP);
(l) monocalcium phosphate anhydrate (MCPA); and
(m) combinations thereof.

15. The kit of claim 14, wherein said peroxyphosphate is selected from the group consisting of:
(a) a peroxymonophosphate;
(b) a diperoxymonophosphate; and
(c) combinations thereof.

16. The kit of claim 15, wherein said peroxymonophosphate is selected from the group consisting of:
(a) monopotassium peroxymonophosphate;
(b) dipotassium peroxymonophosphate;
(c) tripotassium peroxymonophosphate;
(d) monosodium peroxymonophosphate;
(e) disodium peroxymonophosphate;
(f) trisodium peroxymonophosphate;
(g) monoammonium peroxymonophosphate;
(h) diammonium peroxymonophosphate;
(i) triammonium peroxymonophosphate; and
(j) combinations thereof.

17. The kit of claim 15, wherein said diperoxymonophosphate is selected from the group consisting of:
(a) monopotassium diperoxymonophosphate;
(b) dipotassium diperoxymonophosphate;
(c) tripotassium diperoxymonophosphate;
(d) monosodium diperoxymonophosphate;
(e) disodium diperoxymonophosphate;
(f) trisodium diperoxymonophosphate;
(g) monoammonium diperoxymonophosphate;
(h) diammonium diperoxymonophosphate;
(i) triammonium diperoxymonophosphate; and
(j) combinations thereof.

18. The kit of claim 1, wherein said soluble peroxide is selected from the group consisting of:
(a) hydrogen peroxide;
(b) urea peroxide (carbamide);
(c) a peroxyphosphate;
(d) orthophosphate peroxyhydrate;
(e) pyrophosphate peroxyhydrate;
(f) triphosphate peroxyhydrate;
(g) metaphosphate peroxyhydrate;
(h) peroxyborate;
(i) peroxycarbonate;
(j) peroxysilicate; and
(k) combinations thereof.

19. The kit of claim 18, wherein said peroxyphosphate is selected from the group consisting of:
(a) a peroxymonophosphate;
(b) a peroxydiphosphate;
(c) a diperoxymonophosphate; and
(d) combinations thereof.

20. The kit of claim 19, wherein said peroxymonophosphate is selected from the group consisting of:
(a) monopotassium peroxymonophosphate;
(b) dipotassium peroxymonophosphate;
(c) tripotassium peroxymonophosphate;
(d) monosodium peroxymonophosphate;
(e) disodium peroxymonophosphate;
(f) trisodium peroxymonophosphate;
(g) monoammonium peroxymonophosphate;
(h) diammonium peroxymonophosphate;
(i) triammonium peroxymonophosphate; and
(j) combinations thereof.

21. The kit of claim 19, wherein said peroxydiphosphate is selected from the group consisting of:
(a) monopotassium peroxydiphosphate;
(b) dipotassium peroxydiphosphate;
(c) tripotassium peroxydiphosphate;
(d) tetrapotassium peroxydiphosphate;
(e) monosodium peroxydiphosphate;
(f) disodium peroxydiphosphate;
(g) trisodium peroxydiphosphate;
(h) tetrasodium peroxydiphosphate;
(i) monoammonium peroxydiphosphate;
(j) diammonium peroxydiphosphate;
(k) triammonium peroxydiphosphate;
(l) tetraammonium peroxydiphosphate; and
(m) combinations thereof.

22. The kit of claim 19, wherein said diperoxymonophosphate is selected from the group consisting of:
(a) monopotassium diperoxymonophosphate;
(b) dipotassium diperoxymonophosphate;
(c) tripotassium diperoxymonophosphate;
(d) monosodium diperoxymonophosphate;
(e) disodium diperoxymonophosphate;
(f) trisodium diperoxymonophosphate;
(g) monoammonium diperoxymonophosphate;
(h) diammonium diperoxymonophosphate;
(i) triammonium diperoxymonophosphate; and
(j) combinations thereof.

23. The kit of claim 18, wherein said orthophosphate peroxyhydrate is selected from the group consisting of:
(a) sodium orthophosphate peroxyhydrate;
(b) potassium orthophosphate peroxyhydrate;
(c) ammonium orthophosphate peroxyhydrate; and
(d) combinations thereof.

24. The kit of claim 18, wherein said pyrophosphate peroxyhydrate is selected from the group consisting of:
(a) sodium pyrophosphate peroxyhydrate;
(b) potassium pyrophosphate peroxyhydrate;
(c) ammonium pyrophosphate peroxyhydrate; and
(d) combinations thereof.

25. The kit of claim 18, wherein said triphosphate peroxyhydrate is selected from the group consisting of:
(a) sodium triphosphate peroxyhydrate;
(b) potassium triphosphate peroxyhydrate;
(c) ammonium triphosphate peroxyhydrate; and
(d) combinations thereof.

26. The kit of claim 18, wherein said metaphosphate peroxyhydrate is selected from the group consisting of:
(a) sodium metaphosphate peroxyhydrate;
(b) potassium metaphosphate peroxyhydrate;
(c) ammonium metaphosphate peroxyhydrate; and
(d) combinations thereof.

27. The kit of claim 1, wherein said stabilized peroxide composition further comprises a fluoride ion source.

28. The kit of claim 27, wherein said fluoride ion source is selected from the group consisting of:
(a) sodium fluoride;
(b) potassium fluoride;
(c) sodium monofluorophosphate;
(d) sodium hexafluorosilicate; and
(e) combinations thereof.

29. The kit of claim 1, wherein, at 25° C. and at a pH of 7.0, said soluble calcium salt has a calcium ion solubility of at least about 800 ppm, said soluble orthophosphate salt has a phosphate ion solubility of at least about 4000 ppm, and said soluble peroxide has a peroxide solubility of at least about 4000 ppm.

30. A method for whitening and remineralizing teeth, said method comprising the steps of:
(a) combining a first composition comprising a soluble calcium salt with a second composition comprising a soluble peroxide and a soluble orthophosphate salt to yield a mixture, and
(b) during or after said step (a), applying said mixture to said teeth.

31. The method of claim 30, wherein, in said step (b), said mixture is applied using a tray.

32. The method of claim 30, wherein said mixture is applied, in said step (b), after said combining step (a).

33. The method of claim 30, wherein said second composition has a pH from about 2 to about 6.

34. The method of claim 30, wherein said second composition is obtained by combining a pH-control composition having an alkaline pH and a stabilized peroxide composition comprising said soluble peroxide and said soluble orthophosphate salt, wherein said stabilized peroxide composition has an acidic pH.

35. The method of claim 34, wherein at least one of said pH control composition and said stabilized peroxide composition comprises a soluble calcium salt.

36. The method of claim 34, wherein said first composition is an aqueous solution and both of said pH-control composition and said stabilized peroxide compositions are in a gel form.

37. The method of claim 30, wherein said first composition and said second composition, when combined in an aqueous environment, yield a mixture having a pH from about 5 to about 9.

38. A method for whitening and remineralizing teeth, said method comprising the steps of:
(a) applying a first composition comprising a soluble calcium salt to said teeth, and
(b) after said step (a), applying a second composition comprising a soluble peroxide and a soluble orthophosphate salt to said teeth.

39. The method of claim 38, wherein, in said step (b), said second composistion is applied using a tray.

40. The method of claim 38, wherein said second composition has a pH from about 2 to about 6.

41. The method of claim 38, wherein said first composition is an aqueous solution and said second composition is in a gel form.

42. The method of claim 38, wherein said second composition is obtained by combining a pH-control composition having an alkaline pH and a stabilized peroxide composition comprising said soluble peroxide and said soluble orthophosphate salt, wherein said stabilized peroxide composition has an acidic pH.

43. The method of claim 42, wherein at least one of said pH control composition and said stabilized peroxide composition comprises a soluble calcium salt.

44. The method of claim 42, wherein said first composition is an aqueous solution and both of said pH-control composition and said stabilized peroxide compositions are in a gel form.

45. The method of claim 38, wherein said first composition and said second composition, when combined in an aqueous environment, yield a mixture having a pH from about 5 to about 9.

46. A tooth whitening and remineralizing composition comprising solid particles of a soluble calcium salt, a soluble orthophosphate salt, and a soluble peroxide.

47. The composition of claim 46, wherein said composition is in a powder form.

48. The composition of claim 46, wherein said composition is a stable dispersion and further comprises a non-aqueous dispersion medium.

49. The composition of claim 48, further comprising a fluoride ion source.
